(19) United States
(12) Patent Application Publication
(10) Pub. No.: US 2020/0176090 A1
(43) Pub. Date: Jun. 4, 2020

(54) SYSTEMS AND METHODS FOR PATIENT-TRIAL MATCHING

(71) Applicant: Flatiron Health, Inc., New York, NY (US)

(72) Inventors: Achin Batra, Jersey City, NJ (US); Alexander Costet, Brooklyn, NY (US); Alexander Ingram, New York, NY (US); Alexander Padmos, New York, NY (US); Alphan Kirayoglu, New York, NY (US); Brian Shi, New York, NY (US); Dan Ziring, New York, NY (US); Dominic Green, New York, NY (US); Frederick Lindberg, Brooklyn, NY (US); Harvey James Hamrick, JR., Atlanta, GA (US); Janet Donegan, Park City, UT (US); Jeremy Feinstein, Ithaca, NY (US); Jeremy Kohansimeh, New York, NY (US); Jessie Tseng, Brooklyn, NY (US); Lauren Sutton, New York, NY (US); Nathan Chan, New York, NY (US); Rahul Bafna, New York, NY (US); Samuel Helman, Boston, MA (US)

(73) Assignee: Flatiron Health, Inc.

(21) Appl. No.: 16/702,914

(22) Filed: Dec. 4, 2019

Related U.S. Application Data
(60) Provisional application No. 62/775,122, filed on Dec. 4, 2018.
(60) Provisional application No. 62/816,558, filed on Mar. 11, 2019.

(51) Int. Cl. G16H 10/20; G16H 10/60

(52) U.S. Cl. CPC G16H 10/20; G16H 10/60

(57) ABSTRACT

A computer-implemented system for managing electronic medical records may include one or more processors configured to receive, via a user interface of a user device, a user input for adding a new trial and create a new trial portfolio based on the received user input. The portfolio may comprise patient eligibility criteria associated with the new trial. The one or more processors may also be configured to automatically create a patient-trial matching algorithm for the new trial based on the trial eligibility criteria and determine, based on electronic patient medical records associated with a plurality of patients and the patient-trial matching algorithm, at least one suggested patient determined to be eligible for the new trial. The one or more processors may further be configured to transmit, to the user device, instructions for displaying information representing the at least one suggested patient in the user interface.

### CROSS-REFERENCES TO RELATED APPLICATIONS

This application claims the benefit of priority of U.S. Provisional Patent Application No. 62/775,122, filed Dec. 4, 2018, and U.S. Provisional Patent Application No. 62/816,558, filed Mar. 11, 2019, all of which are incorporated herein by reference in their entirety.

### BACKGROUND

### Technical Field

The present disclosure relates to systems and methods for managing electronic medical records.

### Background Information

Identifying patients who are eligible for clinical trials is one of the challenges the cancer research community faces. While there are reasons that may dissuade a patient from participating in clinical trials, there are also many barriers. For example, identifying a patient at just the right time such as, for instance, when they are ready to be put on a therapy but have not yet started one, is often challenging when a practice may have dozens of trials open, each with a dozen or more inclusion/exclusion criteria, and with hundreds of patients coming into a practice a day. Thus, to overcome these challenges faced by existing systems, it is desirable to identify eligible patients for a clinical trial and eligible trials for a patient more efficiently. Additionally, it is desirable to identify the patients scheduled for an office visit who may be eligible for trials to improve trial recruitment, which may benefit both patients and researchers.

### SUMMARY

Embodiments consistent with the present disclosure include systems and methods for providing one or more suggested eligible patients for a clinical trial. Embodiments of the present disclosure may overcome one or more aspects of existing techniques for providing suggested eligible patients for a clinical trial based on computer-generated algorithms according to trial eligibility criteria. The use of computer-generated algorithms in accordance with embodiments of the present disclosure thus allows for faster and more efficient ways for providing patients, physicians, and researchers with reliable suggestions of eligible trials that may benefit the patients.

In one embodiment, a computer-implemented system for managing electronic medical records is provided. The system may include one or more processors configured to receive, via a user interface of a user device, a user input for adding a new trial. The one or more processors may also be configured to create a new trial portfolio based on the received user input, the portfolio comprising patient eligibility criteria associated with the new trial. The one or more processors may further be configured to automatically create a patient-trial matching algorithm for the new trial based on the trial eligibility criteria. The one or more processors may also be configured to determine, based on electronic patient medical records associated with a plurality of patients and the patient-trial matching algorithm, at least one suggested patient determined to be eligible for the new trial. The one or more processors may further be configured to transmit, to the user device, instructions for displaying information representing the at least one suggested patient in the user interface.

In one embodiment, a computer-implemented method for managing electronic medical records is provided. The method may include receiving, via a user interface of a user device, a user input for adding a new trial. The method may also include creating a new trial portfolio based on the received user input, the portfolio comprising patient eligibility criteria associated with the new trial. The method may further include automatically creating a patient-trial matching algorithm for the new trial based on the patient eligibility criteria. The method may also include determining, based on electronic patient medical records associated with a plurality of patients and the patient-trial matching algorithm, at least one suggested patient determined to be eligible for the new trial. The method may further include transmitting, to the user device, instructions for displaying information representing the at least one suggested patient in the user interface.

In one embodiment, a non-transitory computer-readable medium storing instructions for managing electronic medical records is provided. The instructions may be executable by one or more processors to cause the one or more processors to perform a method including receiving, via a user interface of a user device, a user input for adding a new trial. The method may also include creating a new trial portfolio based on the received user input, the portfolio comprising patient eligibility criteria associated with the new trial. The method may further include automatically creating a patient-trial matching algorithm for the new trial based on the patient eligibility criteria. The method may also include determining, based on electronic patient medical records associated with a plurality of patients and the patient-trial matching algorithm, at least one suggested patient determined to be eligible for the new trial. The method may further include transmitting, to the user device, instructions for displaying information representing the at least one suggested patient in the user interface.

### BRIEF DESCRIPTION OF THE DRAWINGS

The accompanying drawings, which are incorporated in and constitute part of this specification, and together with the description, illustrate and serve to explain the principles of various exemplary embodiments. In the drawings:

FIG. 1A is a block diagram illustrating an exemplary system for providing one or more suggested patients for a trial, consistent with the present disclosure.

FIG. 1B is a block diagram illustrating an exemplary computing device for providing one or more suggested patients for a trial, consistent with the present disclosure.

FIG. 2 is a diagram illustrating an exemplary user interface for viewing trials, consistent with the present disclosure.

FIG. 3 is a diagram illustrating an exemplary user interface for receiving user input for creating a new trial, consistent with the present disclosure.

FIGS. 4A and 4B are diagrams illustrating exemplary expression tree structures for one or more suggested patients for a trial, consistent with the present disclosure.

FIG. 5 is a diagram illustrating exemplary expression tree structure for one or more suggested patients for a trial, consistent with the present disclosure.

FIG. 6 is a diagram illustrating an exemplary neural network for providing one or more suggested patients for a trial, consistent with the present disclosure.

FIG. 7 is a diagram illustrating an exemplary user interface for providing one or more suggested trials for patients, consistent with the present disclosure.

FIGS. 8A and 8B are diagrams illustrating an exemplary user interface for providing information of a patient and suggested trials, consistent with the present disclosure.

FIG. 9 is a flowchart illustrating an exemplary process for providing one or more suggested patients for a trial, consistent with the present disclosure.

### DETAILED DESCRIPTION

The following detailed description refers to the accompanying drawings. Wherever possible, the same reference numbers are used in the drawings and the following description to refer to the same or similar parts. While several illustrative embodiments are described herein, modifications, adaptations and other implementations are possible. For example, substitutions, additions or modifications may be made to the components illustrated in the drawings, and the illustrative methods described herein may be modified by substituting, reordering, removing, or adding steps to the disclosed methods. Accordingly, the following detailed description is not limited to the disclosed embodiments and examples. Instead, the proper scope is defined by the appended claims.

Embodiments herein include computer-implemented methods, tangible non-transitory computer-readable mediums, and systems. The computer-implemented methods may be executed, for example, by at least one processor (e.g., a processing device) that receives instructions from a non-transitory computer-readable storage medium. Similarly, systems consistent with the present disclosure may include at least one processor (e.g., a processing device) and memory, and the memory may be a non-transitory computer-readable storage medium. As used herein, a non-transitory computer-readable storage medium refers to any type of physical memory on which information or data readable by at least one processor may be stored. Examples include random access memory (RAM), read-only memory (ROM), volatile memory, non-volatile memory, hard drives, CD ROMs, DVDs, flash drives, disks, and any other known physical storage medium. Singular terms, such as “memory” and “computer-readable storage medium,” may additionally refer to multiple structures, such a plurality of memories and/or computer-readable storage mediums. As referred to herein, a “memory” may comprise any type of computer-readable storage medium unless otherwise specified. A computer-readable storage medium may store instructions for execution by at least one processor, including instructions for causing the processor to perform steps or stages consistent with an embodiment herein. Additionally, one or more computer-readable storage mediums may be utilized in implementing a computer-implemented method. The term “computer-readable storage medium” should be understood to include tangible items and exclude carrier waves and transient signals.

In this disclosure, a system for providing one or more suggested patients for a trial is disclosed.

According to one embodiment, the system may receive user input at a client device for creating a new trial (e.g., a clinical trial) and generate a trial portfolio for the new trial. The system may further obtain or create trial eligibility criteria for the new trial for determining whether a patient is eligible for the new trial. For example, the trial eligibility criteria may include an age requirement, such as an eligible patient must be over 18 years old. As another example, the trial eligibility criteria may include a type of disease (e.g., breast cancer) being treated. The system may also automatically generate a patient-trial matching algorithm for providing one or more suggested patients for the new trial based on the trial eligibility criteria. For example, the system may create a patient-trial matching algorithm based on an expression tree having leaves corresponding to the trial eligibility criteria. The system may further traverse the expression tree using electronic medical records associated with a plurality of patients and determine at least one suggested patient that is likely to be eligible for the new trial.

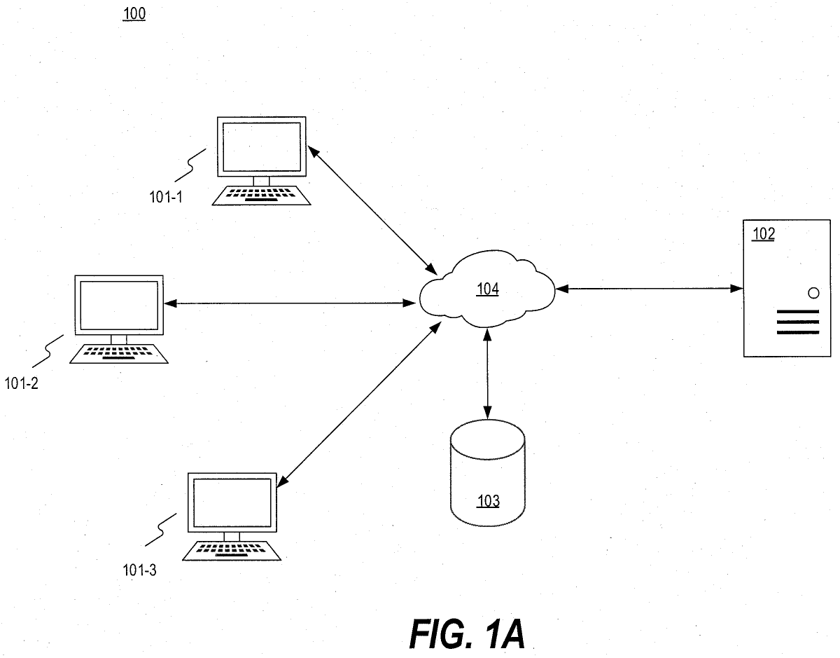

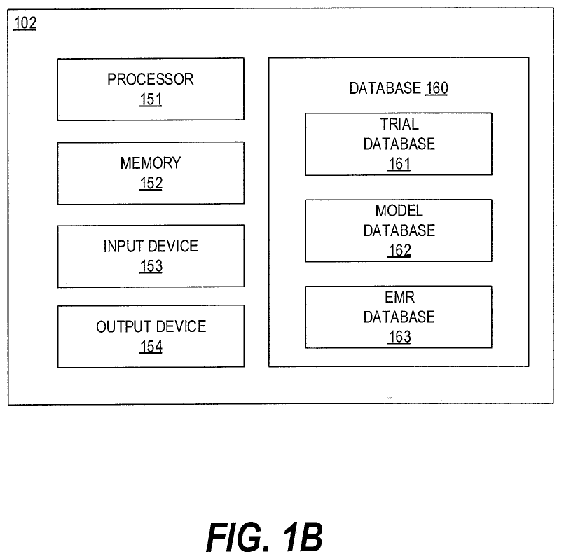

FIG. 1A illustrates an exemplary system 100 for implementing embodiments consistent with the present disclosure, described in detail below. As shown in FIG. 1A, system 100 may include one or more client devices 101, a computing device 102, a database 103, and a network 104. It will be appreciated from this disclosure that the number and arrangement of these components are exemplary and provided for purposes of illustration. Other arrangements and numbers of components may be used without departing from the teachings and embodiments of the present disclosure.

A client device 101 (e.g., client device 101-1, 101-2, 101-3) may be configured to receive user input from a user for creating a new trial. For example, client device 101 may reside at a clinic, and a user (e.g., a physician or administrator) may enter information for creating a new trial portfolio at an input device of client device 101. By way of example, the user may enter an identification number (e.g., a National Clinical Trial (NCT) number or ClinicalTrials.gov identifier) at an interface of client device 101 for creating a new trial, and client device 101 may transmit the identification number to computing device 102. Computing device 102 may create a trial portfolio for the new trial based on the identification number. Client device 101 may also receive and present information received from computing device 102. For example, client device 101 may receive information relating to suggested patients for one or more trials from computing device 102 and present the information at an interface of client device 101 to the user. In some embodiments, client devices 101-1, 101-2, and 101-2 may reside at the same site or different sites.

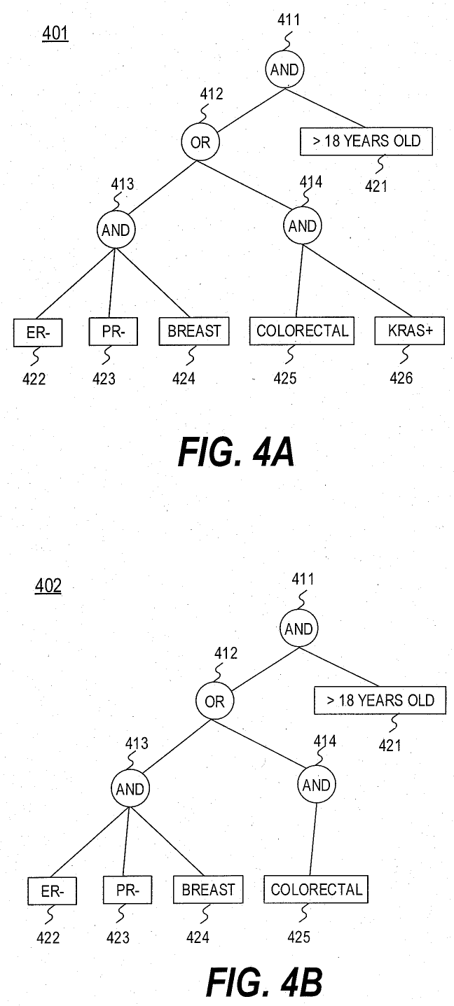

Computing device 102 may be configured to receive information from client device 101 for creating the new trial portfolio from client device 101. Computing device 102 may also create a trial portfolio based on the information received from computing device 102. For example, computing device 102 may receive an NCT number from client device 101 and obtain information relating to the NCT number from a database, which may be an external database (e.g., database 103) or an internal database (e.g., database 160 illustrated in FIG. 1B). The trial information received by computing device 102 may include at least a portion of trial eligibility criteria associated with the trial. Computing device 102 may also create a new trial portfolio for the trial based on the trial information. The trial portfolio may include one or more trial eligibility criteria for determining whether a patient is eligible for the trial. For example, the trial eligibility criteria may include an age restriction that an eligible patent must be over 18 years old. Computing device 102 may further automatically generate an algorithm for suggesting one or more eligible patients for the new trial based on the trial eligibility criteria. For example, computing device 102 may automatically generate an algorithm representing an expression tree (e.g., expression tree structures 401, 402 illustrated in FIGS. 4A and 4B) based on the trial eligibility criteria, and the nodes and/or leaves of the expression tree may represent the trial eligibility criteria. As another example,

Computing device 102 may also be configured to obtain electronic medical records associated with a plurality of patients and determine whether one or more patients may be eligible for the new trial based on the algorithm and electronic medical records. For example, computing device 102 may obtain electronic medical records associated with the patients of a clinical (e.g., the clinical associated with client device 101). Computing device 102 may determine one or more patients among the patients of the clinical who may be eligible for the new trial based on the algorithm and electronic medical records. By way of example, computing device 102 may create a namedtuple that has numbers and a series of letters for each of the patients based on the electronic medical record (e.g., age, disease, biomarkers). Computing device 102 may evaluate the created namedtuples associated with the patients against the expression tree, which may return a number indicating the eligibility for each of the patients. For example, the expression-tree algorithm may output “0” for ineligible or “1” for eligible. Alternatively, the algorithm may output a probability value indicating the eligibility for each of the patients.

Computing device 102 may further be configured to output one or more suggested eligible patients for the new trial. For example, computing device 102 may output one or more suggested patients to an output device (e.g., a display, printer). Alternatively or additionally, computing device 102 may transmit instructions for displaying information representing the one or more suggested patients to client device 101, which may present the information to the user.

In some embodiments, computing device 102 may be configured to provide one or more suggested trials for a patient. For example, the user may select a patient via the input device of client device 101 (or computing device 102), and computing device 102 may provide one or more trials for which the patient may be eligible based on one or more patient-trial matching algorithms and the electronic medical record associated with the patient.

In some embodiments, client device 101 and computing device 102 may be integrated into one device configured to perform the functions of client device 101 and computing device 102 disclosed in this application. For example, a user may input information for creating a new trial via input device 153 of computing device 102, which may display one or more suggested patients for the new trial via an output device (e.g., output device 154, discussed below).

Database 103 may be configured to store information and data for one or more components of system 100. For example, database 103 may store electronic medical records associated with one or more patients. Database 103 may also store information relating to one or more trials. For example, database 103 may store trial eligibility criteria associated with each of the trials. In some embodiments, database 103 may also store patient-trial matching algorithms for determining one or more suggested eligible patients for a trial, and/or one or more suggested eligible trials for a patient. Client device 101 and/or computing device 102 may be configured to access and obtain the data stored on database 103 via network 104. In some embodiments, database 103 may be operated by a third party. For example, computing device 102 may request information relating to a particular trial from database 103, which may transmit the requested information to computing device 102. By way of example, computing device 102 may request the information of trial by transmitting a trial identifier (e.g., an NCT number) to database 103, which may transmit the requested information (e.g., trial eligibility criteria) to computing device 102.

Network 104 may be configured to facilitate communications among the components of system 100. Network 104 may include a local area network (LAN), a wide area network (WAN), portions of the Internet, an Intranet, a cellular network, a short-ranged network (e.g., a Bluetooth™ based network), or the like, or a combination thereof.

FIG. 1B is a block diagram illustrating an exemplary computing device 102. Computing device 102 may include at least one processor (e.g., processor 151), a memory 152, an input device 153, an output device 154, and a database 160.

Processor 151 may be configured to perform one or more functions described in this application. Computing device 102 may also include a memory 152 that may store instructions for various components of computing device 102. For example, memory 152 may store instructions that, when executed by processor 151, may be configured to cause processor 151 to perform one or more functions described herein.

Input device 153 may be configured to receive input from the user of computing device 102, and one or more components of computing device 102 may perform one or more functions in response to the input received. In some embodiments, input device 153 may include an interface displayed on a touchscreen (e.g., output device 154). Output device 154 may be configured to output information and/or data to the user. For example, output device 154 may include a display configured to display one or more suggested patients for a trial. In some embodiments, output device 154 may include a touchscreen.

Database 160 may be configured to store various data and information for one or more components of computing device 102. For example, database 160 may include a trial database 161, a model database 162, and an electronic medical record (EMR) database 163. Trial database 161 may be configured to store information relating to one or more trials. For example, trial database 161 may store a trial portfolio for each of the trials, which may include trial eligibility criteria of a trial. Trial eligibility criteria of a trial may include a trial status, a trial disease, a trial line of therapy, an eligibility age, a trial biomarker criterion, or the like, or a combination thereof. In some embodiments, a trial portfolio may also include trial name, trial description, or the like, or a combination thereof. Trial database 161 may further store edit history including changes made to a trial. Computing device 102 may obtain information relating to the trials from trial database 161 and modify the information if needed. For example, computing device 102 may create a trial portfolio for a new trial and store the trial portfolio into trial database 161.

Model database 162 may store patient-trial matching models or algorithms. A patient-trial matching algorithm refers to an algorithm for determining one or more eligible patients for a trial and/or for determining one or more suggested eligible trials for a patient. Computing device 102 may obtain algorithms from model database 162. In some embodiments, computing device 102 may create an algorithm for a new trial and store the created algorithm into model database 162. EMR database 163 may store electronic medical records associated with patients. Processor 151 may receive one or more electronic medical records from EMR database 163.

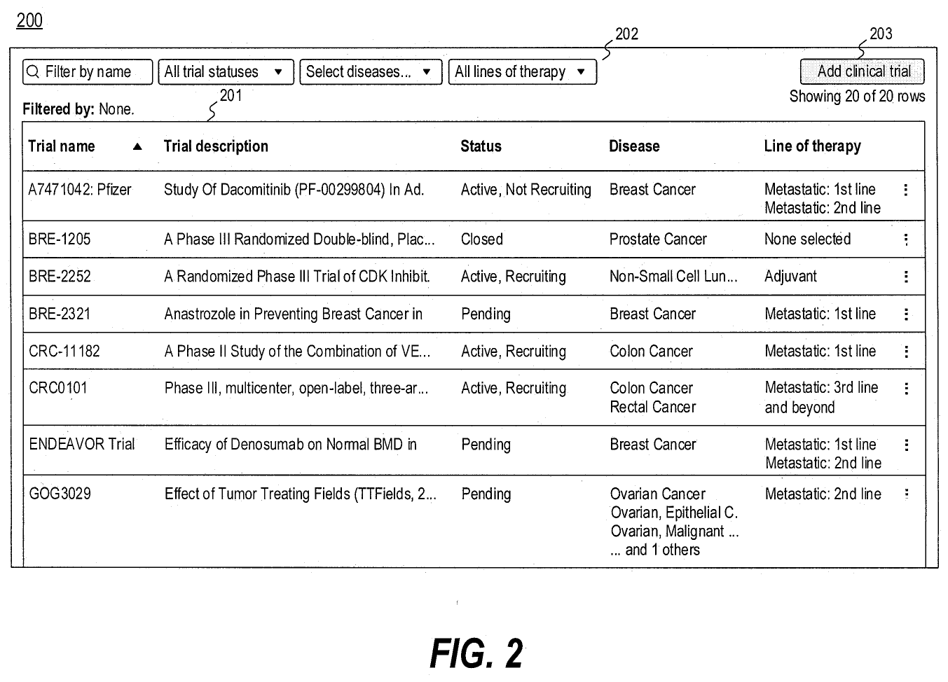

FIG. 2 is a diagram illustrating an exemplary user interface 200 for viewing trials, consistent with the present disclosure. User interface 200 may be displayed via output device 154 of computing device 102 (e.g., a display or touchscreen). Alternatively or additionally, computing device 102 may transmit instructions to client device 101 for displaying user interface 200 via an output device of client device 101 (e.g., a display or touchscreen). Computing device 102 may obtain trial data from a database (e.g., database 103, database 160) and render user interface 200 based on the obtained trial data.

User interface 200 may include a trial list 201. Trial list 201 may include trial name, trial description, trial status, trial disease, trial line of therapy, or the like, or a combination thereof. User interface 200 may also include one or more filters 202, such as filters by trial name, trial description, trial status, trial disease, trial line of therapy, or the like, or a combination thereof. In some embodiments, for each trial, user interface 200 may display the edit history, which may include changes made to the trial information by users at the practice. Thus, users may be able to access one location to see all clinical trials across their practice, the trial status, and the disease and line of therapy that the trial is recruiting. This may help users understand where there may be a gap in their trial portfolio and where they may need to open another trial.

User interface 200 may also include a button 203 for adding a new trial. For example, the user may click or select button 203 (e.g., using a data input device or via selection on a touchscreen), and computing device 102 may render another user interface for the user to enter the information relating to the new trial.

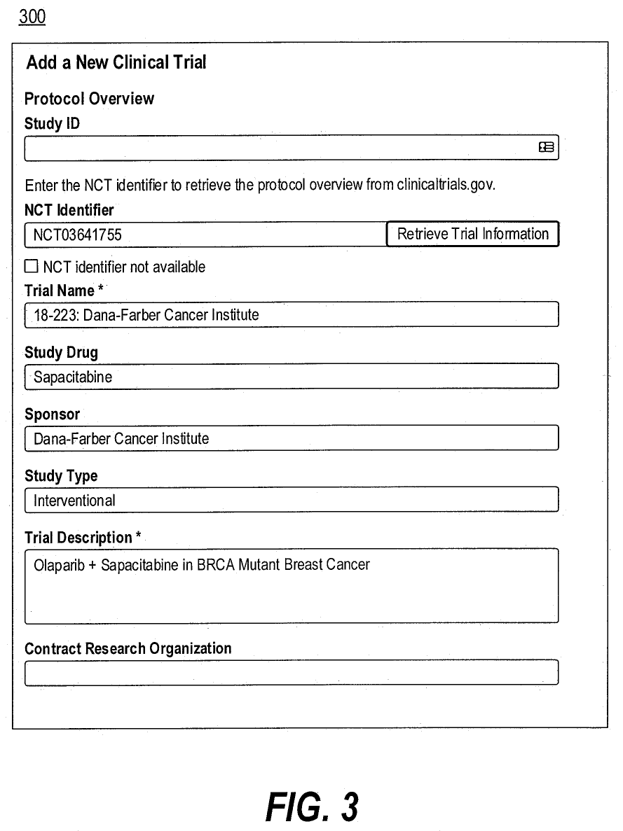

FIG. 3 is a diagram illustrating an exemplary user interface 300 for receiving user input for creating a new trial, consistent with the present disclosure. User interface 300 may be displayed via output device 154 of computing device 102 (e.g., a display or touchscreen), and the user may enter information via input device 153. Alternatively or additionally, computing device 102 may transmit instructions to client device 101 for displaying user interface 300 via an output device of client device 101 (e.g., a display or touchscreen) and for receiving user input via an input device of client device 101.

User interface 300 may include one or more fields for the user to enter to add the new trial. For example, the user may enter a trial identification number. Computing device 102 may obtain or generate a trial based on the trial identification number. By way of example, the user may enter an NCT number, and computing device 102 may obtain the trial information from a database or a third party (e.g., clinicaltrials.gov) based on the received NCT number, including, for example, trial name, study drug, sponsor, study type, trial description, diagnosis, biomarker criteria, line of therapy, or the like, or a combination thereof. Computing device 102 may also populate the information obtained in user interface 300 accordingly. In some embodiments, the user may enter at least a portion of the trial information manually. For example, if an NCT number is not available, the user may check a box to indicate such. User interface 300 may prompt the user to manually complete these fields.

In some embodiments, the trial information may include site information identifying a location where the new trial is to be conducted. For example, the user may enter site information that may help medical practices have oversight into the operations at their practice and receive reporting on their trial performance. The operational data fields may include site ID, principal investigator, trial status, enrollment initiation date, enrollment closing date, institutional review board (IRB) approval date, site initiation visit date, contract execution date, number of days for data entry, enrollment goal (e.g., the number of patients), links to external sources, or the like, or a combination thereof.

Computing device 102 may create a trial portfolio for the new trial store the trial information in database 103 and/or database 160. The trial portfolio may include trial eligibility criteria of the trial. A potential benefit of this approach may be that the user of the system (e.g., an administrator, physician, research coordinator) is able to determine eligible patients at the practice (e.g., a clinical site) against the eligibility criteria. Additionally, the system can provide the user with operational reporting on the trials and patients. This approach may significantly reduce the number of patients that the user needs to review for potential trial eligibility, thereby improving trial recruitment for the practice as the user is spending more time on reviewing patients with a higher likelihood of eligibility.

Computing device 102 may be configured to determine one or more suggested eligible patients for the new trial. For example, computing device 102 may create a patient-trial matching algorithm for determining one or more eligible patients for the new trial based on trial eligibility criteria of the trial. Computing device 102 may also obtain electronic medical records associated with a plurality of patients. For example, computing device 102 may obtain electronic medical records associated with the patients at one or more clinical sites where client device 101 and/or computing device 102 operate from a database (e.g., database 103, database 160). Computing device 102 may further determine one or more eligible patients for the trial based on the electronic medical records and algorithm.

By way of example, the trial eligibility criteria for an example trial may include the following criteria:

1. Is the patient over 18? If yes, go to step 2. If not, they are not eligible.

2. Does the patient have breast cancer? If yes, go to step 3. If not, go to step 4.

3. Is the patient estrogen receptor negative (ER−) and progesterone receptor negative (PR−)? If yes, they are eligible. If not, they are not.

4. Does the patient have colorectal cancer? If yes, go to step 5. If not, they are not eligible.

5. Does the patient have a gene Kirsten rat sarcoma viral oncogene homolog (KRAS) mutation? If yes, they are eligible. If not, they are not.

The above trial eligibility criteria may be represented with Boolean operators, as follows:

(Age >18) AND (ER=Negative AND PR=Negative AND Disease=Breast) OR (KRAS=Positive AND Disease=Colorectal), which may be represented as an exemplary expression tree 401 illustrated in FIG. 4A.

As shown in FIG. 4A, expression tree 401 may include operators 411, 412, 413, and 414, and criteria elements 421, 422, 423, 424, 425, and 426. For example, element 412 may represent that the patient must be over 18 years old. As another example, operators 413 and 414, and elements 422-426 represent that the patient must be either (1) ER negative and PR negative and having breast cancer, or (2) having colorectal cancer and KRAS positive. When computing device 102 evaluates each node, computing device 102 may bubble up the result to the node above it and obtain a result of whether a patient is eligible for this trial. For example, for a patient who has breast cancer and is ER- and PR−, but doesn't have colorectal cancer and hasn't tested KRAS+ may be eligible for the trial because of the left subtree of express tree 401.

In some embodiments, each leaf node in the expression tree represents a single inclusion or exclusion criterion. The nodes (and their criteria) may be mixed and matched into different trees to form the criteria for different trials. Each leaf node may have a role in determining whether a patient is eligible, e.g., taking a patient's clinical information as its input and returning a value that may affect the eligibility as an output. Using an expression tree, the system may enable the user to visualize the matching criteria for a trial and may query various data sources (e.g., electronic medical records of the patients) through a unified interface.

In some embodiments, computing device 102 may be configured to receive updated patient eligibility criteria for the trial. Computing device 102 may also update the patient-trial matching algorithm based on the updated patient eligibility criteria and determine at least one new suggested patient for the updated new trial based on the updated patient-trial matching algorithm and the electronic patient medical records. For example, the user may update the trial eligibility criteria of the trial, and computing device 102 may automatically update the expression tree and patient-trial matching algorithm. As another example, computing device 102 may receive updated trial eligibility criteria from an external database and automatically update the algorithm for the trial based on the updated trial eligibility criteria. By way of example, computing device 102 may receive a user input from the user to delete the KRAS criterium. Computing device 102 may update expression tree 401 by removing leaf node 426 into express tree 402 as illustrated in FIG. 4B. Similarly, if the user adds a new criterium, computing device 102 may insert a new leaf node into the expression tree at an appropriate location. Alternatively or additionally, computing device 102 may modify a leaf node based on input from the user or the system.

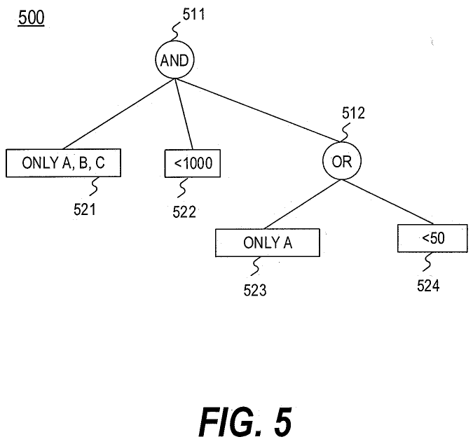

FIG. 5 is a diagram illustrating an exemplary (simplified) expression tree structure, consistent with the present disclosure. FIG. 5 provides an example to further illustrate an expression tree and algorithm. Expression tree 500 may represent trial eligibility criteria including:

(only A, B, C) AND (<1000) AND (only A OR <50).

Expression tree 500 may include operator nodes 511 and 512, which are two operator classes (AND and OR). Operator classes may include children added as leaf classes (e.g., leaves 521, 522, 523, and 524).

Computing device 102 may automatically generate an algorithm representing expression tree 500 based on the trial eligibility criteria. Exemplary code of the algorithm is shown below.

class OrMatchOperator( ):

def init_(self):

self.children = [ ]

def match(self, patient):

prob_no_match = 1.0
for child in self.children:

prob_no_match *= float(1 − child.match(patient))

return 1 − prob_no_match

class AndMatchOperator( ):

def init_(self):

self.children = [ ]

def match(self, patient):

prob_match = 1.0
for child in self.children:

prob match *= float(child.match(patient))

return prob_match

# Mock leaf node that sees whether a MockClass has only certain letters
# in its ‘letters‘ attribute class LetterMatchLeaf( ):

def init_(self, allowable_letters):

self.allowable_letters = allowable_letters

def match(self, patient):

if set(patient.letters) − set(self.allowable_letters):

return 0

return 1

# Mock leaf node that sees whether a MockClass has a number
# less than a max_number class NumberMatchLeaf( ):

def init_(self, max_number):

self.max_number = max_number

def match(self, patient):
return int(patient.number <= self.max_number)

from collections import namedtuple
MockClass = namedtuple(‘MockClass',[‘number’, ‘letters'])
tree = AndMatchOperator( )
tree.children.append(LetterMatchLeaf([‘A’, ‘B’, ‘C’]))
tree.children.append(NumberMatchLeaf(1000))
subtree = OrMatchOperator( )
subtree.children.append(NumberMatchLeaf(50))
subtree, children. append(LetterMatchLeaf([‘A’]))
tree.children.append(subtree)
print(‘Match! (50, [C])’) print(tree.match(MockClass(50, [‘C’]))) print(‘Match! (50, [A])’)
print(tree.match(MockClass(55, [‘A’])))
print(‘Fits neither attribute in the subtree. No match. (50, [C])’)
print(tree.match(MockClass(55,[ ‘C’])))
print(‘Number is too big for the top-level number constraint. No match. (1005,[A])’)
print(tree.match(MockClass(1005,[‘A’])))

The above exemplary code may represent expression tree 500 including the trial eligibility criteria. Computing device 102 may also generate a MockClass for each of the patients, which may be a namedtuple that has a number and a series of letters. For example, computing device 102 may create a namedtuple based on the electronic medical record associated with a patient. The codes also include a leaf class, LetterMatchLeaf, which may only allow a certain subset of letters, and another leaf class, NumberMatchLeaf, which may only allow numbers less than a certain number. One having ordinary skills in the art would understand that these classes are only for illustration purposes and other types of classes may also be used for the algorithm. For example, the algorithm may include a leaf class DiseaseMatchLeaf for disease match and a leaf class BiomarkerMatchLeaf for biomarker match.

Computing device 102 may evaluate different MockClass objects (i.e., the patients) against expression tree 500 using the algorithm, which may return 1 (eligible) or 0 (illegible). In some embodiments, a patient-trial matching algorithm may output a probability, and computing device 102 may determine whether a patient is eligible for the trial based on the probability (e.g., the probability exceeding a threshold).

In some embodiments, computing device 102 may obtain or generate a machine learning algorithm for determining one or more suggested eligible patients for the trial based on the trial eligibility criteria. For example, computing device 102 may obtain a neural network for determining one or more suggested eligible patients for the trial.

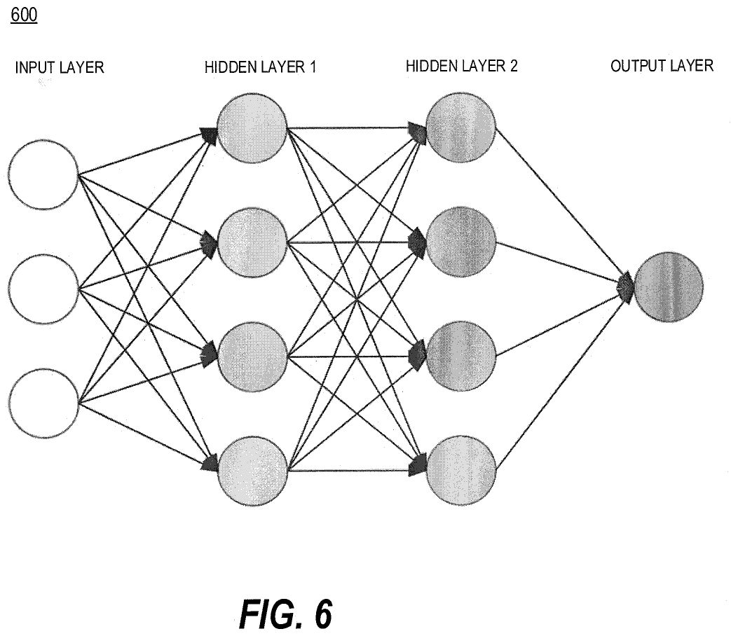

FIG. 6 illustrates an exemplary neural network 600. Neural network 600 may include an input layer, one or more hidden layers, and an output layer. Each of the layers may include one or more nodes. In some embodiments, the output layer may include one node. Alternatively, the output layer may include a plurality of nodes, and each of the nodes may output data. The input layer may be configured to receive input (e.g., an electronic medical record associated with a patient). In some embodiments, every node in one layer is connected to every other node in the next layer. A node may take the weighted sum of its inputs and pass the weighted sum through a non-linear activation function, the results of which may be output as the input of another node in the next layer. The data may flow from left to right, and the final output may be calculated at the output layer based on the calculation of all the nodes. Neural network 600 may output a probability indicating eligibility of the patient for the trial.

In some embodiments, computing device 102 may determine a patient-trial match between a plurality of patients and a plurality of trials, based on the patient-trial matching algorithms associated with the trials and electronic medical records of the patients. For example, computing device 102 may determine one or more suggested eligible patients for each of the trials and/or one or more suggested eligible trials for each of the patients. Computing device 102 may also generate a data structure representing the relationship between the patients and trials and store the data structure in a database (e.g., database 103, database 160). Computing device 102 may further present the data representing the relationship between the patients and trials to the user. For example, computing device 102 may be configured to generate a patient-trial matching report. By way of example, computing device 102 may receive user input for defining filters for the data to appear on the report, including, for example, patient information (e.g., gender, age, location, patient schedule, diagnosis, biomarker, or the like, or a combination thereof), treatment information (e.g., treatment, inclusionary and/or exclusion drug), and trial information (trial name, study drug, sponsor, study type, trial description, diagnosis, biomarker criteria, line of therapy, or the like, or a combination thereof). Computing device 102 may compile the patients and/or trials that match the filtered data into a report.

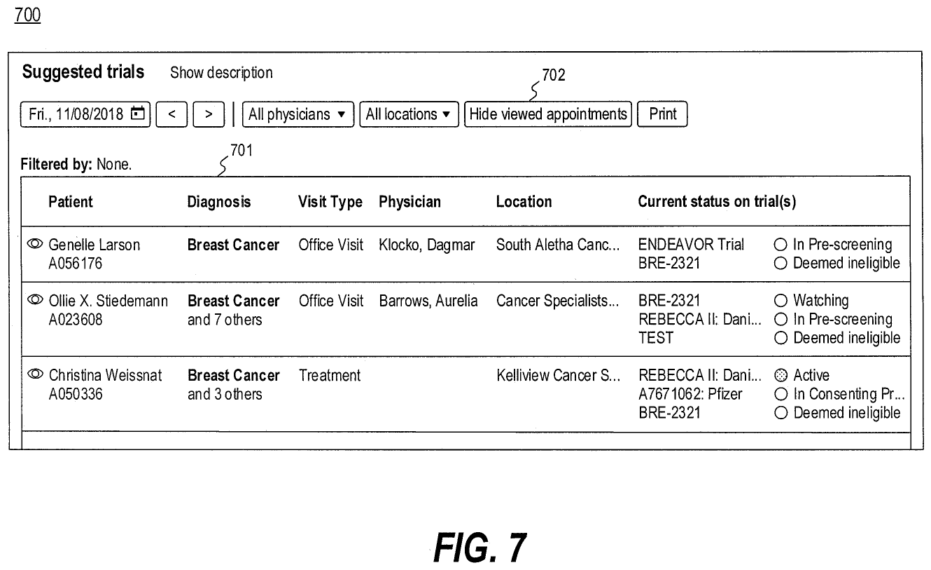

FIG. 7 is a diagram illustrating an exemplary user interface for providing one or more suggested trials for patients, consistent with the present disclosure. User interface 700 may be displayed via output device 154 of computing device 102 (e.g., a display or touchscreen), and the user may enter information via input device 153. Alternatively or additionally, computing device 102 may transmit instructions to client device 101 for displaying user interface 700 via an output device of client device 101 (e.g., a display or touchscreen) and for receiving user input via an input device of client device 101.

The user may select a patient schedule, and computing device 102 may access the patient schedule and determine the patients who have been scheduled for a visit according to the patient schedule. For example, the user may select a date (e.g., Nov. 8, 2018 shown in FIG. 700), and computing device 102 may access a patient schedule and determine the patients who have been scheduled for a visit on that date. Alternatively or additionally, the user may view a patient schedule for a period (e.g., a week, a month). Computing device 102 may also provide the user with an interface that shows information relating to the patients and visits. For example, as illustrated in FIG. 7, user interface 700 may include the patients' names, diagnoses, visit types (e.g., office visit, treatment), physicians who the patients visit, locations of the visits, or the like, or a combination thereof.

Computing device 102 may further determine one or more suggested trials for the patients based on the algorithm associated with the trials and the electronic medical records of the patients as described elsewhere in this disclosure. Computing device 102 may also represent user interface 700 to the user, including a list of suggested trials for the patients. As illustrated in FIG. 7, user interface 700 may include a patient list 701, which may include the information of the patients, such as each patient's name, diagnosis, visit type, trial, patient's status, or the like, or a combination thereof. User interface 700 may also include filters 702 configured to receive the user's input to filter patients and/or trials according to, for example, physician, location, patient diagnosis, visit type, trials, patient status, or the like, or a combination thereof. By presenting suggested trials for the patients who have been scheduled for a visit, the patient-trial matching may be tied directly to the patient schedule of the practice (e.g., a clinical) so that the user of the system can identify eligible patients who are visiting the clinical on a particular date and can schedule meetings with these patients to discuss a potential opportunity to participate in the trials. This may improve the patient recruitment. For example, computing device 102 may provide the user with one or more suggested eligible patients who will visit a clinic on a particular date. As another example, the user may filter the trials and/or patents according to diseases, type of trials, or the like, or a combination thereof. Computing device 102 may inform the physician and/or research coordinator to discuss with the patient about the trial in which the patient may be eligible for participation. For example, computing device 102 may include the trial information into the patient's medical record so that the physician may be reminded when discussing with the patient.

In some embodiments, user interface 700 may display a patient schedule including a doctor appointment of at least one suggested patient. Alternatively or additionally, user interface 700 may display information of a doctor or a location associated with the doctor appointment of the patient.

In some embodiments, computing device 102 may update user interface 700 according to the user's input. For example, if a patient name or patient record has been selected (e.g., clicked into), user interface 700 may show that the patient name or patient record appears as “viewed” (e.g., displaying a “viewed” icon by the name of the patient or by another patient identifier). User interface 700 may include a filter to filter the patient(s) who have been viewed.

In some embodiments, user interface 700 may also include different views according to the user's preferences. For example, user interface 700 may include a “Suggested Trials” view, as shown in FIG. 7, which may display the patients with suggested trials (e.g., actively recruiting or pending trials that match the patient(s)'s diagnosis and biomarkers). Alternatively or additionally, user interface 700 may include a “New Patients” view (not shown), which may display patients who are new to the practice and are having their first visit to the practice. Alternatively or additionally, user interface 700 may include a “Recent Updates” view, which may display patients with suggested trials or who were previously marked as “candidate” or “watching” and should be considered (or reconsidered) now because of a recent scan or pathology report. As another example, recent updates may include a recent scan such as, for example, a pathology or scan report received or a scan order created in the electronic health record associated with a patient since his or her last office visit.

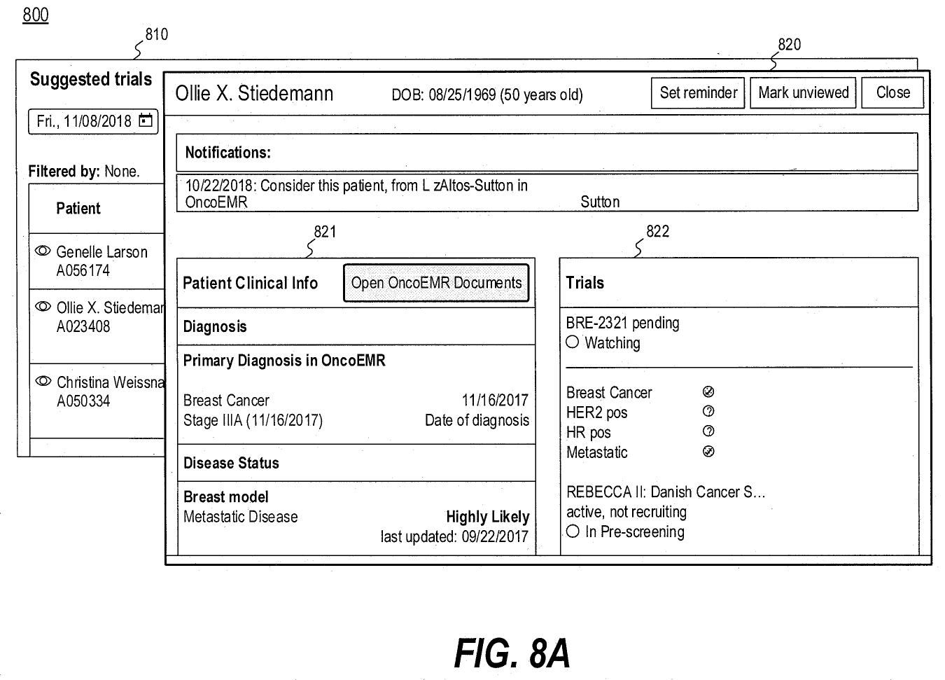

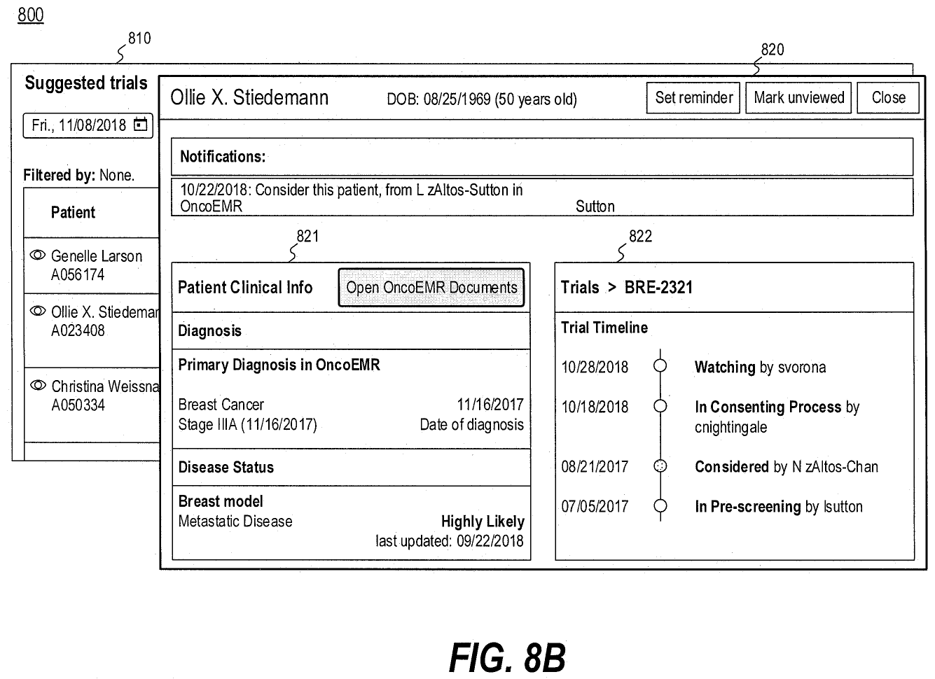

In some embodiments, when the user clicks or selects a patient name in user interface 700, computing device 102 may process the input and provide another user interface for displaying the information of the patient to the user. For example, FIGS. 8A and 8B are diagrams illustrating an exemplary user interface 800 for providing information of a patient and suggested trials, consistent with the present disclosure. User interface 800 may be displayed via output device 154 of computing device 102 (e.g., a display or touchscreen), and the user may enter information via input device 153. Alternatively or additionally, computing device 102 may transmit instructions to client device 101 for displaying user interface 800 via an output device of client device 101 (e.g., a display or touchscreen) and for receiving user input via an input device of client device 101.

As illustrated in FIG. 8A, a user may click or select the patient “Ollie X. Sitedemann” in user interface layout 810 (which is similar to user interface 700). Computing device 102 may provide the user with a user interface layout 820, which may partially overlap with user interface layout 810, for displaying the information of the patient. By way of example, user interface layout 820 may include a region 821 displaying the clinical information of the patient, including, for example, patient's diagnosis information, last office visit, disease, or the like, or a combination thereof. In some embodiments, user interface layout 820 may allow the user to open the electronic medical record of the patient. User interface layout 820 may also include a region 822 displaying one or more suggested and/or existing trials for the patient, which may include the trial information, such as trial name, trial description, trial status, trial disease, trial line of therapy, or the like, or a combination thereof. In some embodiments, user interface 800 may display information associated with two or more trials, which may include the statuses of the trials.

In some embodiments, user interface 800 may present more detailed information regarding a trial. For example, the user may select a trial named “BRE-2321” in region 822, and computing device 102 may update region 822 of user interface 800 for displaying more information of the trial, as illustrated in FIG. 8B. For example, region 822 may be updated to display a trial timeline of the trial.

In some embodiments, user interface 800 may allow the user to take an action on the information of patient and/or one or more of the trials. For example, user interface 800 may allow the user to update the information (e.g., marking the patient as unviewed). The updated information may be displayed on user interface 800 accordingly. In some embodiments, the updated information may be saved for further use and/or be made available for another user of the system. For example, when a first user has viewed a patient (by, for example, clicking or selecting in user interface 800), computer device 102 may label the patient as “viewed.” Computing device 102 may also provide a second user with an user interface including an indicator indicating that this patient has been viewed. Alternatively or additionally, user interface 800 may allow the user to set up a reminder for the user, physician, or research coordinator, or the like, or a combination thereof, to visit the information. By way of example, user interface 800 may allow the user to create a reminder for a physician who has been scheduled to see the patient to look into potentially eligible trials.

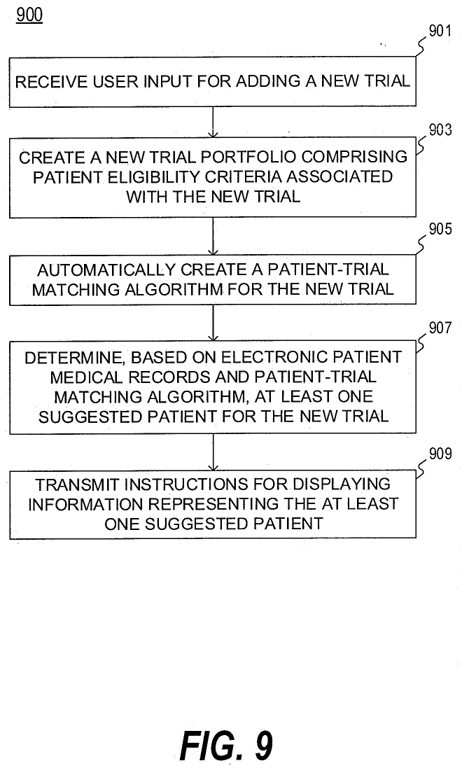

FIG. 9 is a flowchart illustrating an exemplary process 900 for providing one or more suggested patients for a trial, consistent with the present disclosure. While process 900 is described in connection with computing device 102, one skilled in the art would understand that one or more steps of process 900 may be performed by other components of the system (e.g., client device 101 or processor 151).

At step 901, computing device 102 may receive user input via a user interface of computing device 102 or client device 101 for creating a new trial, as described in this disclosure. By way of example, the user may enter an identification number (e.g., an NCT number or ClinicalTrials.gov identifier) at an interface of client device 101 for creating a new trial (e.g., user interface 300 illustrated in FIG. 3). Client device 101 may transmit the identification number to computing device 102. Computing device 102 may obtain trial information from a database (e.g., database 103, database 160) based on the identification number. In some embodiments, the user interface may allow the user to enter trial information in one or more fields, as illustrated in FIG. 3. For example, the user may enter the trial information, such as trial name, study drug, sponsor, study type, trial description, diagnosis, biomarker criteria, line of therapy, or the like, or a combination thereof; via user interface 300.

At step 903, computing device 102 may be configured to create a new trial portfolio based on the user input and/or trial information, as described in this disclosure. In some embodiments, the trial portfolio may include trial eligibility criteria associated with the trial.

At step 905, computing device 102 may be configured to automatically create a patient-trial matching algorithm for determining one or more eligible patients for the new trial based on the trial eligibility criteria, as described elsewhere in this disclosure. For example, computing device 102 may automatically create a patient-trial matching algorithm representing an expression tree (e.g., expression tree 401 illustrated in FIG. 4A) or neutral network (e.g., neural network 600 illustrated in FIG. 6) based on the trial eligibility criteria.

At step 907, computing device 102 may be configured to determine, based on electronic patient medical records associated with a plurality of patients and the patient-trial matching algorithm, at least one suggested patient determined to be eligible for the new trial, as described in this disclosure. For example, computing device 102 may generate a nametuple for each of the patients based on electronic medical records associated with the patient, which may include a number and a series of letters. Computing device 102 may evaluate the nametuples against an expression tree using the algorithm, which may return 1 (eligible) or 0 (illegible). Computing device 102 may determine whether a patient is eligible for the trial based on the returned value by the algorithm.

At step 909, computing device 102 may be configured to transmit, to client device 101 or output device 154, instructions for displaying information representing the at least one suggested patient in the user interface, as described in this disclosure. For example, computing device 102 may transmit to client device 101 instructions for displaying user interfaces 700 and 800 illustrated in FIGS. 7, 8A, and 8B.

The foregoing description has been presented for purposes of illustration. It is not exhaustive and is not limited to the precise forms or embodiments disclosed. Modifications and adaptations will be apparent to those skilled in the art from consideration of the specification and practice of the disclosed embodiments. Additionally, although aspects of the disclosed embodiments are described as being stored in memory, one skilled in the art will appreciate that these aspects can also be stored on other types of computer readable media, such as secondary storage devices, for example, hard disks or CD ROM, or other forms of RAM or ROM, USB media; DVD, Blu-ray, 4K Ultra HD Blu-ray, or other optical drive media.

Computer programs based on the written description and disclosed methods are within the skill of an experienced developer. The various programs or program modules can be created using any of the techniques known to one skilled in the art or can be designed in connection with existing software. For example, program sections or program modules can be designed in or by means of .Net Framework, .Net Compact Framework (and related languages, such as Visual Basic, C, etc.), Java, Python, R, C++, Objective-C, HTML, HTML/AJAX combinations, XML, or HTML with included Java applets.

Moreover, while illustrative embodiments have been described herein, the scope of any and all embodiments having equivalent elements, modifications, omissions, combinations (e.g., of aspects across various embodiments), adaptations and/or alterations as would be appreciated by those skilled in the art based on the present disclosure. The limitations in the claims are to be interpreted broadly based on the language employed in the claims and not limited to examples described in the present specification or during the prosecution of the application. The examples are to be construed as non-exclusive. Furthermore, the steps of the disclosed methods may be modified in any manner, including by reordering steps and/or inserting or deleting steps. It is intended, therefore, that the specification and examples be considered as illustrative only, with a true scope and spirit being indicated by the following claims and their full scope of equivalents.

What is claimed is:

1. A computer-implemented system for managing electronic medical records, comprising:
one or more processors configured to:
receive, via a user interface of a user device, a user input for adding a new trial;
create a new trial portfolio based on the received user input, the portfolio comprising patient eligibility criteria associated with the new trial;
automatically create a patient-trial matching algorithm for the new trial based on the trial eligibility criteria;
determine, based on electronic patient medical records associated with a plurality of patients and the patient-trial matching algorithm, at least one suggested patient determined to be eligible for the new trial; and
transmit, to the user device, instructions for displaying information representing the at least one suggested patient in the user interface.

2. The system of claim 1, wherein the one or more processors are further configured to receive at least a portion of the trial eligibility criteria from an external database based on the received input.

3. The system of claim 2, wherein the one or more processors are further configured to receive the portion of the trial eligibility criteria based on a trial identifier included in the received input.

4. The system of claim 1, wherein the trial eligibility criteria includes at least one of a trial status, a trial disease, a trial line of therapy, an eligibility age, or a trial biomarker criterion.

5. The system of claim 1, wherein displaying the information representing the at least one suggested patient in the user interface comprises displaying a patient schedule including a doctor appointment of the at least one suggested patient.

6. The system of claim 5, wherein displaying the patient schedule comprises displaying information of a doctor or a location associated with the doctor appointment of the at least one suggested patient.

7. The system of claim 1, wherein displaying the information representing the at least one suggested patient in the user interface comprises displaying information representing one or more other trials for the at least one suggested patient.

8. The system of claim 7, wherein displaying the information representing the one or more other trials comprises displaying a status of the one or more other trials.

9. The system of claim 1, wherein the one or more processors are further configured to:
receive updated patient eligibility criteria for the new trial;
update the patient-trial matching algorithm based on the updated patient eligibility criteria; and
determine at least one new suggested patient for the updated new trial based on the updated patient-trial matching algorithm and the electronic patient medical records.

10. The system of claim 1, wherein displaying the information representing the at least one suggested patient comprises:
displaying patent information of the at least one suggested patient in a first portion of the user interface; and
displaying information identifying the new trial in a second portion of the user interface.

11. A computer-implemented method for managing electronic medical records, comprising:
receiving, via a user interface of a user device, a user input for adding a new trial;
creating a new trial portfolio based on the received user input, the portfolio comprising patient eligibility criteria associated with the new trial;
automatically creating a patient-trial matching algorithm for the new trial based on the patient eligibility criteria;
determining, based on electronic patient medical records associated with a plurality of patients and the patient-trial matching algorithm, at least one suggested patient determined to be eligible for the new trial; and
transmitting, to the user device, instructions for displaying information representing the at least one suggested patient in the user interface.

12. The method of claim 11, further comprising receiving at least a portion of the patient eligibility criteria from an external database based on the received input.

13. The method of claim 12, further comprises receiving the portion of the patient eligibility criteria based on a trial identifier included in the received input.

14. The method of claim 11, wherein the patient eligibility criteria includes at least one of a trial status, a trial disease, a trial line of therapy, an eligibility age, or a trial biomarker criterion.

15. The method of claim 11, wherein displaying the information representing the at least one suggested patient in the user interface comprises displaying a patient schedule including a doctor appointment of the at least one suggested patient.

16. The method of claim 15, wherein displaying the patient schedule comprises displaying information of a doctor or a location associated with the doctor appointment of the at least one suggested patient.

17. The method of claim 11, wherein displaying the information representing the at least one suggested patient in the user interface comprises displaying information representing one or more other trials for the at least one suggested patient.

18. The method of claim 17, wherein displaying information representing the one or more other trials comprises displaying a status of the one or more other trials.

19. The method of claim 11, further comprises:
receiving updated patient eligibility criteria for the new trial;
updating the patient-trial matching algorithm based on the updated patient eligibility criteria; and
determining at least one new suggested patient for the updated new trial by based on the updated patient-trial matching algorithm and the electronic medical records of the plurality of patients.

20. A non-transitory computer-readable medium comprising instructions that when executed by one or more processors, cause the one or more processors to:
receive, via a user interface of a user device, a user input for adding a new trial;
create a new trial portfolio based on the received user input, the portfolio comprising patient eligibility criteria associated with the new trial;
automatically create a patient-trial matching algorithm for the new trial based on the patient eligibility criteria;
determine, based on electronic patient medical records associated with a plurality of patients and the patient-trial matching algorithm, at least one suggested patient determined to be eligible for the new trial; and
transmit, to the user device, instructions for displaying information representing the at least one suggested patient in the user interface.
